(19) United States
(12) Patent Application Publication
(10) Pub. No.: US 2010/0197027 A1
(43) Pub. Date: Aug. 5, 2010

(54) AN INDICATING FIBER

(76) Inventors: Yifan Zhang, Woodbury, MN (US); Jie J. Liu, Woodbury, MN (US); Eric M. Moore, Roseville, MN (US); Francis E. Porbeni, Woodbury, MN (US); Scott J. Tuman, Woodbury, MN (US); Diane R. Wolk, Woodbury, MN (US)

Correspondence Address: 3M INNOVATIVE PROPERTIES COMPANY, PO BOX 33427, ST. PAUL, MN, 55133-3427, US

(21) Appl. No.: 12/666,047

(22) PCT Filed: Jun. 25, 2008
(86) PCT No.: PCT/US08/68094

§ 371 Date: Dec. 22, 2009

Related U.S. Application Data
(60) Provisional application No. 60/966,560, filed on Jun. 29, 2007.

(51) Int. Cl. G01N 33/72; G01N 33/68; C08K 5/07; G01N 33/48; D01D 5/00; H05B 7/00

(52) U.S. Cl. 436/66; 436/86; 524/357; 436/94; 264/484

(57) ABSTRACT

A fiber, an articles formed from a fiber and methods of making the fiber and associate article are disclosed. In one embodiment, the fiber comprises a synthetic polymer and a color-changing indicator. The color-changing indicator is dispersed throughout the synthetic polymer. The color-changing indicator reacts in the presence of a stimulus to produce a color change.

### FIELD

The present invention relates to a reacting indicating fiber, articles constructed from an indicating fiber, and a method of making an indicating fiber.

### BACKGROUND

Fibers are used throughout industry to make a variety of products such as fabrics, wipes, and scouring articles. The fibers may be formed from natural materials, like cotton, or from synthetic materials, like thermoplastic resins or reconstituted cellulose. Fibers made from thermoplastic resins are particularly useful in making nonwoven articles. An example of a nonwoven article made from thermoplastic fibers is a Scotch-Brite® Scouring Pad, available from 3M of St. Paul, Minn. A variety of nonwoven articles made from polymer-based fibers and can range from highly stiff to very drapeable articles to serves a variety of purposes, particularly cleaning purposes.

The polymer fibers can be made in a variety of sizes from a variety of known processing techniques, which can result in microfibers and nanofibers. Microfibers and nanofibers can be used to make articles, such as nonwoven articles. Microfibers and nanofibers have very small diameters, which result in an article formed from a microfiber or nanofiber having a very large surface area. For cleaning purposes, the large surface area helps to capture and retain dirt, debris, and oil from soiled surfaces. One example of a thermoplastic microfiber article is a Scotch-Brite® Kitchen Cloth, available from 3M of St. Paul, Minn.

Articles formed from polymer-based fibers can be made from a variety of known processing methods. Typically, these processing methods result in the ability to make fibers and articles from those fibers inexpensively. Therefore, articles formed from polymer fibers are suitable as disposable articles, and particularly as disposable cleaning articles.

For cleaning applications, such as in kitchens and bathrooms, biological contaminants or microorganisms such as e. coli or salmonella may be present. If the user cleanes the surface with a wipe, the user may have spread the soil or may have captured it. However, in either situation, the user is unable to ascertain the true cleanliness of the surface following cleaning. For providing a clean environment, the user needs an indication of the cleanliness of a surface.

### SUMMARY

A fiber, articles formed from a fiber, and methods of making the fiber and associated article are disclosed. The fiber contains a color-changing indicator that is capable of giving a visual indication in response to a stimulus. In one embodiment, the visual indication is representative of the cleanliness of the surface being wiped.

In one embodiment, a fiber is disclosed that comprises a synthetic polymer and a color-changing indicator. The color-changing indicator is dispersed throughout the synthetic polymer. The color-changing indicator reacts in the presence of a stimulus to produce a color change.

In one embodiment, an article for indicating the presence of a substance on a surface comprises a plurality of fibers, wherein at least a portion of the fibers are color-indicating fibers comprising a synthetic polymer, a color-changing indicator dispersed throughout the synthetic polymer that reacts in the presence of a stimulus to produce a color change on a working surface of the article. In an embodiment, the article may be a woven article, knitted article, or a nonwoven article.

In one embodiment, a method of making a fiber comprises providing a synthetic polymer, providing a color-changing indicator, dispersing the color-changing indicator throughout the synthetic polymer, and forming a fiber. In one embodiment, forming the fiber may be from melt blowing, spunbond, melt spinning, dry spinning, wet spinning and gel spinning, or electrospinning.

### DETAILED DESCRIPTION

A fiber, articles formed from a fiber, and methods of making the fiber and associated article are disclosed. The fiber contains a color-changing indicator that is capable of giving a visual indication in response to a stimulus. The fiber comprises a synthetic polymer and a color-changing indicator.

In one embodiment, the synthetic polymer of the indicating fiber comprises a thermoplastic material. Suitable thermoplastic materials include, but are not limited to, polyesters, polyamides, polyimides, nylon, polyolefins (e.g., polypropylene and polyethylene), poly(ethylene vinyl alcohol) copolymer (PEVOH), poly(propylene vinyl alcohol) copolymer (PPVOH), polylactic acid (PLA), or combinations thereof. In another embodiment, the synthetic polymer of the fiber comprises regenerated cellulose, including rayon.

A “color changing indicator” is one or more chemical compounds that will interact with a stimulus to produce a visually discernable color change. The stimulus may be pH, protein, amine, sugar including glucose, or hemoglobin/myoglobin to give a reaction for the particular color-changing indicator. Typically, the stimulus will be associated with a particular contaminant. For example, if the color-changing indicator responds to amino groups, then the color-changing indicator will respond to a protein. Protein is present in meat. Meat products such as beef can carry e. coli and chicken can carry salmonella. Therefore, a color-changing indicator that responds to an amino group may indicate meat protein is present and contaminations, such as e. coli or salmonella, may be present.

In one embodiment, the color-changing indicator will give a visually discernable color change within 15 minutes under room temperature conditions. Depending on the particular use of the fiber it may be desirable to achieve a visually discernable color change within 5 minutes or further within 2 minutes.

The color-changing indicator is dispersed throughout the synthetic polymer. Therefore, at a cross-section of an indicating fiber, the color-changing indicator is dispersed across the cross-section. This is distinguished from a fiber that make be treated with a dye after being formed so that the dye is essentially coated on the surface of a fiber and not dispersed throughout. In one embodiment, the color-changing indicator is uniformly dispersed throughout the synthetic polymer. Therefore, at a cross-section of an indicating fiber, the color-changing indicator is uniformly dispersed throughout the indicating fiber. In one embodiment, the color-changing indicator may be present from 0.1 to 15 wt % of the fiber. In a further embodiment, the color-changing indicator may be present from 1 to 10 wt % of the indicating fiber.

In one embodiment, the color-changing indicator includes a functional group and is a functionalized color changing indicator. A “functionalized color changing indicator” is a color changing indicator with a functional group capable of forming covalent bonds to a reactive group of the synthetic polymer. As described above, the functionalized color changing indicator may be dispersed throughout the synthetic polymer and covalently bond with the synthetic polymer. The functionalized color changing indicator covalently bonded to the synthetic polymer can be further processes as described below in the same manner a non-functionalized color changing indicator may be processed. U.S. Patent Application 60/947,030, filed on Jun. 29, 2007 titled “Ninhydrin Functionalized Polymer,” the disclosure of which is herein incorporated by reference, discloses ninhydrin functionalized polymers that may be suitable to be processed into a fiber.

One suitable color-changing indicator is ninhydrin that chemically reacts in the presence of amino acids, amines and amino sugars to form a vivid purple product called Ruhemann's Purple. Therefore, ninhydrin can detect a protein by reacting to the amino group of the protein. Ninhydrin is commercially available in a hydrate formation as triketohydrindane hydrate, 2,2-dihydroxy-1,3-indandione. At room temperature, the hydrate is a stable, pale yellow, slightly hygroscopic crystalline powder. In certain solutions, the ketone 1,2,3-Indantrione may be present in less than 3%. The reaction is shown below:

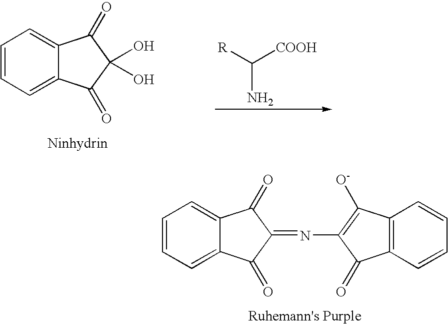

In one embodiment, a functionalized ninhydrin, shown below, may be incorporated in to the synthetic polymer having reactive hydroxyl groups, such a PVA, and further processed as described below and made into articles as described below so long as the ability of the color changing indicator to produce a color change in the presence of a stimulus is maintained.

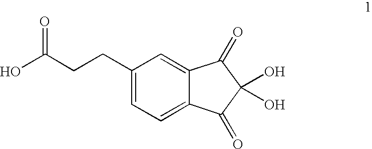

Another color-changing indicator that may be suitable is a bicinchoninic acid (BCA) assay. For BCA assay, copper sulfates (CuSO₄) reacts with protein at basic conditions to reduce the Cu²⁺ ion to Cu⁺. Then, the Cu⁺ ion complexes with BCA to form a purple colored complex. The Bradford Assay (Coomassie Brilliant Blue G-250), Lowry Assay, Biuret Assay, all capable of giving color changes in the presence of a protein, may be incorporated into the components used to form a fiber. Further, hemoglobin and glucose detection systems may be used. One hemoglobin system is 3,3′5,5′-tetramethylbenzidine (TMB) and cumen hydroperoxide in a buffer solution. Another hemoglobin system is 3-methyl-2-benzothiazolinone hydrazone hydrochloride monohydrate (MBTH), 3-(dimethylamino)benzoic acid (DMAB), and hydrogen peroxide (H₂O₂) in a buffer solution. Other hemoglobin system are benzidine, o-tolidine, o-toluidine, and o-dianisidine each in a peroxide system in a buffer solution. The MBTH/DMAB hemoglobin detection system can be modified by adding glucose oxidase and peroxidase such as horseradish to be used to detect glucose. Other systems can be used to detect glucose, such as KI/glucose oxidase/peroxidase. It is believed that these, along with others, can be incorporated into the components used to form the fiber.

In embodiments where the indicating fiber, and ultimately the article made from the indicating fiber, is used on surfaces that come into contact with people or pets, the color-indicator chosen should be safe and nontoxic. Other additives may be included in the fiber. Additives such as, but not limited to, adhesives, anti-oxidants, dyes, pigments, surfactants, soaps, detergents, anti-microbial agents and fiber finishes may be present in the fiber.

The fibers may be made in a variety of known processing techniques to make fibers ranging in size, shape, and length. It is within the scope to make nanofibers and microfibers. Nanofibers provide a particularly unique indicating fiber. An article comprised of nanofibers generally has a large surface area. It is believed that this property will allow for a faster reaction time of the color-changing indicator because the color-changing indicator is readily available to react to the stimulus. Further, due to the processing of the nanofiber the color-changing indicator may be more integrally formed as part of the fiber.

To make an indicting fiber, which comprises a color-changing indicator, a variety of known processing techniques may be used. One process is referred to as melt blowing. In a typical melt-blown process, pellets or otherwise solid materials are introduced to an extruder where the blend is heated and then introduced to the melt-blown die. While melt-blowing is usually done with thermoplastic polymers, the process may also be used to form polymeric solutions into fibers. In a melt blown process for making an indicating fiber, a solid form of the color-changing indicator may be mixed with the dry materials prior to entry in to an extruder or a liquid form of the color-changing indicator may be added directly to the extruder. Alternatively, the color-changing indicator may be compounded in high concentrations before the fiber forming process. This pre-compounded masterbatch would then be introduced into the process, along with un-compounded material to produce a fiber with the desired color-changing indicator concentration.

In the process, the color-changing indicator and other materials are heated, blended, and incorporated with the synthetic polymer. It is desirable to select the synthetic polymer and color-changing indicator, and other materials if necessary, so that the color-changing indicator is relatively compatible with the synthetic polymer. Having the color-changing indicator compatible with the synthetic polymer is believed to lock the color-changing indicator into the formed indicating fiber and prevent the color-changing indicator from bleeding out and separating from the synthetic polymer of the indicating fiber and ultimately onto the surface being wiped. Addition of a surfactant may result in a more compatible solution for the color-changing indicator to disperse into the synthetic polymer. Also, the color-changing indicator should be capable of withstanding the conditions of the melt-blowing process.

Spunbond is another process for making fibers that may be used in making indicating fibers. U.S. Pat. No. 3,338,992 discloses a method of making spunbond fibers. Because spunbond is similar to the melt blown process, the color-changing indicator, in a solid state or liquid, is introduced to the extruder, blended, and incorporated with synthetic polymer prior to processing the indicating fiber. Because a synthetic polymer solution is utilized, the same considerations regarding compatibility and the color-changing indicator withstanding processing conditions are relevant when making an indicating fiber with the spunbond process.

Continuous indicating fibers may be spun by a number of different methods including melt spinning, dry spinning, wet spinning and gel spinning. Descriptions of commonly practiced long fiber forming techniques can be found in Fundamentals of Fibre Formation, by Andrzej Ziabicki. Generally, these processes extrude a fiber from fluid, either melt or solution, through a die. The color-changing indicator may be added to the fluid as discussed above with respect to the melt-blown process. The extrudate is drawn from the die, pulling the extrudate into a fiber. During the drawing process, the fiber forming material is solidified through some combination of cooling, drying, or chemical reaction. The solidified fibers are then wound up or carried on for further processing. After spinning, fibers may be subjected a variety of post processing steps. Examples of post processing include crimping, cutting, dying, heat-setting, post-drawing, coating, and twisting.

Electrospinning is another process that may be used for making indicating fibers. Electrospinning is particularly applicable in making fibers of very small diameter, such as nanofibers. U.S. Pat. No. 1,975,504 discloses a process of electrospinning. A fiber-forming liquid is formed containing the synthetic polymer, color-changing indicator, and optionally a solvent or other processing aid. The fiber forming liquid used for electrospinning may be either a molten liquid or a liquid containing a substance that will solidify into a fiber form during the electrospinning process. Electrospinning generally involves the creation of an electrical field at the surface of a liquid. The resulting electrical field draws the fiber forming fluid into a stream that is drawn towards a grounded collector. As the jet of fiber forming fluid elongates and travels, it will solidify. The solidification of the fiber forming fluid is accomplished through cooling, solvent evaporation, or chemical reaction, or some combination thereof. The fibers are collected either directly on the grounded collector or a substrate placed in the path of the fiber forming fluid. The fibers may be used on a substrate or collector directly, or removed for further processing or use.

When utilizing a solution for electrospinning, the color-changing indicator, synthetic polymer, and processing solvent should be chosen so that the color-changing indicator and synthetic polymer are both dispersible in the solvent and therefore are able to be dispersed prior to the electrospinning process.

In one particular embodiment, electrospun fiber can be made from poly(ethylene vinyl alcohol) copolymer (PEVOH), poly(propylene vinyl alcohol) copolymer (PPVOH), polylactic acid (PLA). Solvents that can be used with these polymers are isopropyl alcohol, water, H₃PO₄, CHCl₃ and DMF and combinations thereof. Examples of solvents that can be used include IPA/H₂O (70/30), H₂O/H₃PO₄ (99/1), H₂O, and CHCl₃/DMF (4/1). Generally, most polymer solutions may be electrospun.

Another method of making and webs including nanofibers is disclosed in U.S. Pat. Nos. 6,183,670 and 6,315,806 to Torobin et al. Torobin et al. discloses the following ('670 patent, Summary of the Disclosure, col. 7, line 43 to col. 8, line 58):
Preferred embodiments of the present invention relate to methods and apparatuses for producing composite fibrous media composed of discontinuous fine fibers and discontinuous ultra-fine electrostatically charged or uncharged fibers. Further preferred embodiments relate to composite fibrous media produced thereby and filtration media, particle wipe media and absorbent media comprising such composite fibrous media.
Preferred embodiments employ a source of fiberizing gas and a source of molten polymer fluid substance or substances which, when combined with a jet stream of fiberizing gas, will produce filaments of the polymer as it cools. Preferred embodiments of an apparatus include a cell mounting plate, in which is mounted a planar array of a plurality of rows of fiber producing cells, each cell capable of adjustably controlling the diameter and angle of spray of a mixture of molten polymer and fiberizing gas, a plurality of conduits supplying the molten polymer fluid and fiberizing gas to the fiber producing cells, a foraminous belt, a plurality of belt driver rolls, a moveable air permeable collection surface such as screen mesh, an air suction box, and a plurality of compaction rolls.
Filtration medium is made, preferably, by a two dimensional array of equally spaced and individually adjustable cells, each of which is supplied with fiberizing gas and molten polymer to produce a single high velocity two-phase solids-gas jet of discontinuous fibers entrained in air. The individual cells in the array are rotatably positioned relative to each other so that the jet spray from a cell is induced to intermingle and combine with the jet sprays of neighboring cells in its proximity. This enhances the collision and consequential intermingling and intertwining of nascent fibers in flight in the region of fiber formation, in a manner which causes the fibers to deform and become entangled with and partially wrap around each other at high velocity and in a localized fine scale manner before they have had an opportunity to cool to a relatively rigid state.
The collided and entangled fibers are subsequently formed into a web by being drawn onto the upper surface of a planar section of a moving continuous foraminous belt by means of an air flow induced by a high air volume suction box placed in contact with the underside of the section of the belt.
Preferably, the cells are individually adjusted to control the mean diameters, lengths and trajectories of the fibers they produce. Certain cells in the two dimensional array may be adjusted to generate a significant percentage of fibers having diameters less than one micron diameter, and which are relatively shorter in length. Certain other cells in the array may be adjusted to generate a significant percentage of structure-forming reinforcing fibers having diameters greater than one micron diameter which are relatively longer in length. By employing appropriately close positioning and orientation of the cells in the array, drag forces created by air eddies induced by the colliding sprays of adjacent cells are used to induce sub-micron diameter fibers to partially entwine around some of the larger diameter fibers. The sub-micron fibers are thereby caused to promptly entangle with and partially wrap around the larger reinforcing fibers. The larger fibers thereby trap and immobilize the sub-micron diameter fibers in a fine scale manner in the region of their formation to minimize the tendency of sub-micron diameter fibers to clump, agglomerate, or rope together in flight. Also, the cells producing the larger fibers are selected to form a protective curtain of larger fibers around each cell producing sub-micron diameter fibers, to prevent the sub-micron diameter fibers from being carried off by stray air currents, or to subsequently to detach from their position in the settled web. The entangled larger fibers also overcome the inherent mechanical weakness and excessive compressibility of sub-micron fiber webs, thereby enabling the practical use of sub-micron fibers in filtration systems, including air filtration systems.
The resultant aggregate of commingled and intertwined fibers are subsequently deposited on a moving air permeable collection surface such as a composite fibrous web. The fiber aggregate is drawn down and compacted onto the air permeable moving collection surface by negative air pressure induced by the suction box. In further preferred embodiments the resultant aggregate is compacted by passing the aggregate through compaction rollers.

Through use of the Torobin process, the color-changing indicator may be included in some or all of the molten polymer fluid to produce indicating fibers that contain the color-changing indicator. It is understood that not all of the produced fibers must include the color-changing indicator. Also, as discussed above with respect to melt-blown methods of making fibers the particular color-changing indicator chosen should be compatible with the polymer fluid of the melt and able to withstand the temperatures experienced during the fiber-forming process.

Making multilayer or multicomponent fibers is known. U.S. Pat. Nos. 5,176,952; 5,238,733; 5,258,220; 5,207,970; and 5,232,770 disclose multi-layer fibers and various uses for such multi-layer fibers. It is understood that an indicating fiber according to the present invention may be a multilayer fiber, wherein one or more of the layers includes the color-changing indicator.

It is understood that a variety of fiber length, diameters, sizes, shapes and constructions may be made in accordance with the teachings of the present invention. In one embodiment, a nanofiber is formed. A nanofiber is understood to be a fiber with a diameter less than 1 micron. In one embodiment, a microfiber is formed. A microfiber is understood to be a fiber with a diameter larger than a nanofiber but less than 1 denier (approximately 20 microns). For commonly used non-woven fibers, a fiber of 1 denier is typically between 10 and 15 microns in diameter. In another a fiber is formed that has a diameter larger than a microfiber. In one embodiment, the fiber has a length of at least 1 mm. In another embodiment, the fiber has a length that is essentially endless, as understood by one skilled in the art.

The indicating fiber typically will be formed into an article prior to use. Articles may be made from weaving, knitting, and nonwoven processes. To make a nonwoven a variety of processes are known including carding, garneting, airlaying, spunbond, wet-laying, melt blowing, stitchbonding. Further processing of a nonwoven may be necessary to add properties such as strength, durability, and texture. Examples of further processing include calendering, hydroentangling, needletacking, resin bonding, thermobonding, ultrasonic welding, embossing, and laminating.

The nonwoven article may be comprised entirely from color-indicating fibers or from a blend of color indicating fibers and other fibers, which may be polymer based, natural fibers or metal fiber. Additionally, the color-indicating fibers may be arranged in a specific pattern. It is known that different types of fibers may be blended together to make an article. The mixing of the fibers may be done integrally with another process or separately from any fabric, web, or yarn forming process.

The article can have any size, shape, or rigidity depending on the end use needs. Coatings of materials such as resins, surfactants, detergents, which may or may not include abrasive particles may be placed over the article. The coatings should be applied in a way so as not to inhibit the ability of the color-changing indicator to give a color response. For example, resin may be spot coated to specified areas of the articles and not to the entire article. The article may be a layered product comprising various layers of different combinations of nonwoven, woven or knitted materials, film, foam, sponge, and various combinations thereof. If layered, the layers may be laminated, stitched, needlepunched or otherwise bonded to secure the layers together. Some or all of the layers may have indicating fibers. Some of the layers may not have any indicating fibers.

The article may be provided in a wet or dry state. The article itself may be absorbent or may have absorbent layers secured to the article. In a wet state, the article may be saturated with solutions of water, alcohols, detergents, surfactants, or disinfectants, or combinations thereof so long as the solution does not adversely affect the color-changing indicator or the color-changing indicator's ability to give a color change in the presence of the stimulus. Disinfectants may be particularly suitable for an article intended for cleaning purposes. Common surface disinfectants comprise biocides such as alcohols, biguanides, cationic surfactants, and halogen or halogen containing compounds. Suitable alcohols include ethanol and isopropyl alcohol (IPA) in 70% water [IPA/H₂O (70/30), EtOH/H₂O (70/30)]. Suitable biguanide (chlorhexidine) are polyhexamethylene biguanide, p-chlorophyenyl biguanide, and 4-chlorobenzhydryl biguanide. Commercially available biguanides are Nolvasan® available from Wyeth of Fort Dodge, Iowa and ChlorhexiDerm® Disinfectant available from DVM Pharmaceuticals of USA. Examples of cationic surfactant (Quaternary Ammonium Compounds, Quats) include Parvosol® available from Hess & Clark of Randolph, Wis., Roccal-D® Plus available from Pfizer of New York, N.Y., Unicide™ 256 available from Brulin & Coompany Inc. of Indianapolis, Ind., benzalkonium chloride. Typical halogen or halogen containing compounds are either chlorine or iodine based.

To use an article formed from a coloring-indicated fiber, the article is passed over a surface. If the surface is free of a stimulus capable of giving a color-change with the color-changing indicator, then no visual color change is apparent. Then, the user knows the surface is essentially free of that stimulus. Typically, the stimulus will be associated with a particular contaminant. Therefore, the user knows the surface is essentially free of the associated contaminant.

If the surface includes the stimulus that is capable of giving a color-change with the color-changing indicator, then a visual color change will appear. The users know the surface includes the stimulus and the associated contaminant.

In one embodiment, the color-changing indicator is responsive to a protein stimulus through reaction with an amine group. Therefore, a color change in the color-changing indicator within the indicating fiber is indicative of protein on the surface, which may be indicative of bacteria such as e. coli or salmonella being present on the surface.

In the embodiment where the article further includes a disinfectant, a wipe across the surface to detect a color change will also deliver a portion of the disinfectant. Therefore, upon seeing a color change some of the disinfectant will act upon the stimulus on the surface. The user may wipe the surface again with a new article to determine if the stimulus had been removed.

### EXAMPLES

### Preparation of Stimulus

Protein solutions, generally referred to as meat juice, were prepared. Approximately 10 grams of fresh pork chop meat was extracted with 20 mL of water for 16 hours and the mixture was filtered. The total protein in the meat juice was measured according to Pierce assay. The total protein content for the meat juice was approximately 11 mg/mL.

### Preparation of Fiber

Two processing conditions were performed: melt-blowing and electrospinning

Melt blown webs were produced using a 38 mm conical twin screw extruder, feeding a gear-type positive displacement pump, which then fed the melt blowing die. The melt-blowing die was of a drilled orifice type using 0.015 inch (0.318 mm) diameter holes, and 25 holes per inch of die width. The die had a nominal web width of 10 inches (25.4 cm). Drilled orifice melt blowing dies are disclosed by Harding, Buntin, and Keller in U.S. Pat. No. 3,825,380. The web was collected using a screened vacuum collector, and rolled up from the surface of the collector. In the table below, the melt extrusion temperature and the resulting web basis weight in grams per square meter is noted.

Electrospinning was accomplished using a typical laboratory needle-based electrospinning unit. The polymer was dissolved in solvent prior to spinning, then loaded into a syringe. At the end of the syringe was a flat-tipped stainless steel hypodermic needle. The syringe was placed into a syringe pump (Model 22, From Harvard Apparatus, Holliston, Mass.) to provide constant flow. The grounded target used was an aluminum weighing dish clamped to a ring stand. The distance between the tip of the syringe needle and the grounded target is referred to as the target distance. An adjustable high voltage power supply was connected to the needle and grounded target to produce the desired electric field. When the syringe pump and high voltage supply are both activated, a fiber stream is carried from the needle to the grounded target. The spinning process was allowed to continue for a known amount of time sufficient to generate a nonwoven web coating on the grounded target. Fiber diameters were then measured using scanning electron microscopy.

Fiber Size

SEM (Scanning Electron Microscope) was used to measure the fiber diameters of the nanofibers. SEM photomicrographs were taken of each web at 1K and 10K magnifications using a LEO VP 1450 SEM (15 kV, 15 mm WD, 0° tilt, gold/palladium-coated sample under high vacuum). Fiber diameter measurements of at least 25 individual fibers were taken from the images at 10,000 time magnification, and an average fiber diameter was calculated.

For the melt-blown webs and effective fiber diameter (EFD) was measured. Fiber diameter is taken from the pressure drop of a known air flow through the web. This method for estimating average fiber diameter is discussed in Davies, C. N., “The Separation of Airborne Dust and Particulates,” Inst. Mech. Eng., London, Proceedings 1B, 1952.

Product List

Ninhydrin available from Aldrich Chemical Co. of Milwaukee, Wis.

Polypropylene vinyl alcohol (PPVOH), 57K-66K (86-89% hydrolyzed) available from Alfa Aesar of Ward Hill, Mass.

Polyethylene vinyl alcohol (PEVOH), EVAL C109B available from Kuraray of Houston, Tex.

Polylactic acid (PLA), PLA 6251D available from NatureWorks LLC of Minneapolis, Minn.

Polypropylene (PP), Exxon 6936 PP available from ExxonMobil of Houston, Tex.

Reaction Measurement

The prepared indicating fibers were exposed to the prepared meat juice at room temperature. A visual inspection was conducted to determine when a noticeable visual color change in the indicating fiber occurred. The time in minutes was measured and is noted in Table 1 below.

TABLE 1

| | | | | | Fiber | |
|---|---|---|---|---|---|---|
| | | | Color-changing | | Size | Reaction |
| Example | Polymer | Solvent | indicator | Processing | (Avg.) | time (min) |
| 1 | PEVOH | Isopropanol:H₂O | Ninhydrin | Electrospinning: | 0.49 | 4 |
| | 8 wt % | (70:30 wt %) | (5 wt %) | Voltage: 15 KV | microns | |
| | | | | Target D: 18 cm | | |
| | | | | Coat time: 3 min. | | |
| 2 | PPVOH | H₂0:H₃PO₄ | Ninhydrin | Electrospinning: | 0.56 | 10 |
| | 10 wt % | (99:1 wt %) | (3 wt %) | Voltage: 20 KV | microns | |
| | | | | Target D: 18 cm | | |
| | | | | Coat time: 40 min. | | |
| 3 | PPVOH | H₂O (100 wt %) | Ninhydrin | Electrospinning: | 0.42 | 10 |
| | 10 wt % | | (3 wt %) | Voltage: 20 KV | microns | |
| | | | | Target D: 18 cm | | |
| | | | | Coat time: 20 min. | | |
| 4 | PLA | DMF:CHCl₃ | Ninhydrin | Electrospinning: | 0.42 | 6 |
| | 10.5 wt % | (1:4 wt %) | (3 wt %) | Voltage: 20 KV | microns | |
| | | | | Target D: 18 cm | | |
| | | | | Coat time: 5 min. | | |
| 5 | PLA | DMF:CHCl₃ | Ninhydrin | Electrospinning: | 1.10 | 6 |
| | 12.5 wt % | (1:4 wt %) | (3 wt %) | Voltage: 20 KV | microns | |
| | | | | Target D: 18 cm | | |
| | | | | Coat time: 5 min. | | |
| 6 | PP | | Ninhydrin | Blown Microfiber | 13.2 | 11 |
| | (95 wt %) | | (5 wt %) | Ext. temp: 200° C. | microns | |
| | | | | Web basis: 59 gsm | | |

Although specific embodiments of this invention have been shown and described herein, it is understood that these embodiments are merely illustrative of the many possible specific arrangements that can be devised in application of the principles of the invention. Numerous and varied other arrangements can be devised in accordance with these principles by those of ordinary skill in the art without departing from the spirit and scope of the invention. Thus, the scope of the present invention should not be limited to the structures described in this application, but only by the structures described by the language of the claims and the equivalents of those structures.

What is claimed is:

1. A fiber comprising:
a synthetic polymer;
a color-changing indicator;
wherein the color-changing indicator is dispersed throughout the synthetic polymer; and
wherein the color-changing indicator reacts in the presence of a stimulus to produce a color change.

2. The fiber of claim 1, wherein the synthetic polymer is selected from the group consisting of a thermoplastic polymer and regenerated cellulose.

3. The fiber of claim 1, wherein the color-changing indicator produces a color change as a result of a chemical reaction.

4. The fiber of claim 1, wherein the color-changing indicator responds to a protein stimulus.

5. The fiber of claim 4, wherein the color-changing indicator is ninhydrin.

6. The fiber of claim 1, wherein the color-changing indicator is uniformly dispersed throughout the synthetic polymer and therefore dispersed throughout a cross-section of the fiber.

7. The fiber of claim 1, wherein the color changing indicator comprises 0.1 to 15 wt % of the fiber.

8. The fiber of claim 1, wherein the fiber has a diameter of less than 250 microns.

9. The fiber of claim 1, wherein the stimulus is selected from the group consisting of a pH condition, protein, amine, hemoglobin, sugar, and carbohydrate.

10. The fiber of claim 1, wherein a plurality of fibers are arranged to form an article.

11. An article for indicating the presence of a substance on a surface comprising:
a plurality of fibers, wherein at least a portion of the fibers are color-indicating fibers comprising a synthetic polymer, a color-changing indicator dispersed throughout the synthetic polymer that reacts in the presence of a stimulus to produce a color change on a working surface of the article.

12. The article of claim 11, wherein the plurality of fibers are interconnected.

13. The article of claim 11, wherein the plurality of fibers form a nonwoven web.

14. The article of claim 11, wherein the article is bonded to a backing.

15. The article of claim 14, wherein the backing comprises a woven, knitted, or nonwoven fabric, sponge, foam, film, or paper.

16. The article of claim 11, wherein the color-changing indicator is ninhydrin and reacts in the presence of a protein.

17. The method of making a fiber comprising:
providing a synthetic polymer;
providing a color-changing indicator;
dispersing the color-changing indicator throughout the synthetic polymer;
forming a fiber.

18. The method of claim 17, further comprising forming the fiber from a melt.

19. The method of claim 18, further comprising arranging a plurality of melt-formed fibers to form a nonwoven article.

20. The method of claim 17, wherein forming the fiber comprises electrospinning.
